SEQUENCE LISTING (4 sequences)

SEQ ID NO: 1 - DNA, 20 bp, Artificial Sequence
  Description of Artificial Sequence Synthetic sense primer
gagtttggatcttggttcat

SEQ ID NO: 2 - DNA, 22 bp, Artificial Sequence
  Description of Artificial Sequence Synthetic antisense primer
ggcacgggcagcttgccggtgg

SEQ ID NO: 3 - DNA, 23 bp, Artificial Sequence
  Description of Artificial Sequence Synthetic sense primer
ttttcctggatcatgcctggcac

SEQ ID NO: 4 - DNA, 21 bp, Artificial Sequence
  Description of Artificial Sequence Synthetic anitsense primer
ctacctgtagcagcttaccca

(19) United States
(12) Patent Application Publication
(10) Pub. No.: US 2005/0155093 A1
(43) Pub. Date: Jul. 14, 2005

(54) Rabbit nuclear cloning method and uses thereof

(75) Inventors: Patrick Chesne, Voisins Le Bretonneux (FR); Pierre Adenot, Morsang Sur Orge (FR); Jean-Paul Renard, Vanves (FR)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Institute National de Ia Recherche Agronomique, Paris (FR)

(21) Appl. No.: 10/501,065

(22) PCT Filed: Jan. 10, 2003
(86) PCT No.: PCT/FR03/00064

(30) Foreign Application Priority Data
Jan. 10, 2002 (FR) 02 00268

(51) Int. Cl. A01K 67/027

(52) U.S. Cl. 800/14; 800/15; 800/16; 800/17; 800/18

(57) ABSTRACT

The invention concerns a method for producing non-human mammal embryos, in particular rabbit by nuclear cloning. The invention also concerns the mammals obtained and their uses.

### BACKGROUND OF THE INVENTION

This application claims the benefit of the filing date of French Application No. 02/04821, filed on Apr. 17, 2002, the disclosure of which is hereby incorporated herein by reference.

The present invention relates to a method for nuclear cloning of vertebrates, in particular of mammals, and more particularly of rabbits. The invention also relates to the animals thus produced, in particular rabbits, at the fetal or adult stage, and to their use for the production of molecules of interest or as animal models for studying human pathologies.

Rabbits are increasingly considered in the biotechnological industry for the advantages which they offer compared with other animal species. First of all, from a phylogenic point of view, the rabbit is closer to primates, that is to say humans, than rodents, mice and rats, which are commonly used at the present time (Graur et al., 1996). Next, the rabbit is better suited to physiological manipulation by virtue of its size, unlike small animals such as rodents, mice and rats, or large animals such as cows, goats, sheep, pigs and the like. Finally, the large size of the litter and a rapid reproduction are other advantages for a laboratory animal intended for the study of human pathologies or for the production of recombinant proteins of interest. Thus, for example, the rabbit model is of great interest for the development of a clinical treatment of arteriosclerosis (Hoeg et al., 1996) and of cystic fibrosis (Chen et al., 2001).

Somatic nuclear transfer associated with genetic modifications of nucleus-donating cells could render extremely advantageous the use of rabbits as a laboratory animal (Fan et al., 1999) which, at the moment, is confined to the production of recombinant proteins of interest in large quantity (Stinnackre et al., 1997). The technology of nuclear transfer is of great interest because it allows the rapid production of a large number of genetically identical animals, or their progeny, which have specific genetic characteristics. However, to date, it has never been possible to carry out nuclear transfer in rabbits successfully (Yin et al., 2000; Dinnyés et al., 2001) despite the pioneering role of rabbits in the experiments for developing the nuclear cloning technology (Bromhall et al., 1975).

A single research laboratory has been able to obtain the start of gestations of embryos from somatic cell nuclei (Yin et al., 2000), but surprisingly, none of these gestations were able to reach full term.

The rabbit, although initially used as animal model for the development of nuclear transfer techniques (Bromhall et al., 1975) therefore appears as a species for which the existing nuclear cloning technologies, which have been successful with other species such as sheep (Wilmut et al., 1997; WO 97 07669), mice (Wakayama et al., 1998; WO 99 37143), bovines (Wells et al., 1999), goats (Baguisi et al., 1999; WO 00 25578), pigs (Polejaeva et al., 2000), are not suitable. A great need therefore exists to develop a novel method for nuclear cloning of animal species which, up until now, cannot be efficiently cloned by the nuclear transfer methods currently available, and in particular to develop a method for nuclear cloning in rabbits, which is efficient, reproducible and which makes it possible to obtain good cloning yield.

### SUMMARY OF THE INVENTION

That is the problem which the present invention proposes to solve by providing a method for producing mammalian embryos comprising the following steps:
(a) evaluate and/or determine the asynchrony of development (T) between two embryos of the same species and of the same age:
   the first embryo being produced by crossing at the time t₀ a male, preferably vasectomized, with a female who has preferably received hormone treatment to increase ovulation, the said first embryo being at least cultured and/or manipulated in vitro;
   the second embryo being produced by crossing at the time t₀ a fertile male with a female who has preferably received hormone treatment in order to increase ovulation, said second embryo being normally fertilized and obtained by parthenogenetic activation,
the evaluation and/or determination taking place at the latest on the day of uterine implantation of said second embryo, and;
(b) transfer an embryo which is at least cultured and/or manipulated in vitro into the uterus of a recipient female who was crossed with a vasectomized male at the time t=t₀+T(±25% T);
(c) optionally, allow said embryo transferred in step b) to become implanted and to develop in the uterus of said recipient female.

In principle, the present invention is applicable to all mammals. More particularly, the animal according to the invention is a mammal. The invention is particularly advantageous for mammals such as hoofed animals, equine animals, members of the camel family, rodents, lagomorphs and primates. Among the rodents, there may be mentioned mice, rats, hamsters and guinea pigs. Among the hoofed animals, there may be mentioned bovines, ovines, caprines and porcines. Preferably, the mammal according to the invention is a lagomorph. The invention is particularly advantageous for animals which, up until now, were difficult or even impossible to obtain by nuclear cloning, such as rabbits or rats.

### DETAILED DESCRIPTION

The expression “asynchrony” is understood to mean, for the purposes of the present invention, the time or delay expressed in hour which exists in a given instant of embryonic development between the stage of development of an embryo normally fertilized and developing with the laws of nature, and the stage of development of an embryo which, at a given moment of its development has at least been manipulated in vitro, the two embryos being of the same age and of the same species. The expression embryos of the same age is understood to define that the embryos were conceived simultaneously or at the same time. Thus, in the case of a normally fertilized embryo and a reconstituted embryo obtained by nuclear transfer, the enucleated oocyte will have the same age as the oocyte normally fertilized by a sperm. The two embryos can belong to the same animal species but also to different species. In the case of nuclear cloning in rabbits, the two embryos are rabbit embryos. These two embryos may or may not be of different breeds such as the “New Zealand”, “Fauve-de-Bourgogne”, “Argenté-de-Champagne”, Californian, “Géant-de-Bouscat” breed, or any breed whose zoological specificities are defined in an official standard (Le lapin de race, Ed. 2000 Fédération Frangaise de Cuniculture (Ed.)) or obtained from crossings which have given rise to commercial strains of rabbits such as the GD22/1077 strain.

The expression “cultured in vitro” is understood to mean, for the purposes of the present invention, an embryo which is not conceived and/or developed naturally, that is to say an embryo for which at least one stage of its conception and/or of its development is carried out in vitro. For example, the embryo “cultured in vitro” for the purposes of the present invention is cultured and develops in an appropriate culture medium containing the nutritive elements necessary for the growth and/or differentiation of the embryo.

“Manipulated in vitro” is understood to mean, for the purposes of the present invention, an embryo cultured in vitro, obtained by nuclear transfer and/or genetically modified by transgenesis. The embryo is cultured and/or manipulated in vitro at the latest up to the day preceding the implantation.

According to the present invention, the evaluation and/or the determination of asynchrony is carried out at the latest on the day of uterine implantation of the embryo fertilized normally or obtained by parthenogenic activation or by cloning; nevertheless, this determination of asynchrony is preferably carried out at a stage of development chosen from the 1 cell stage, the 2 cell stage, the 4 cell stage, the 8 cell stage, the 16 cell stage, the morula stage and the blastocyte stage. Preferably, the evaluation and/or the determination of asynchrony is carried out using embryo(s) having reached the blastocyte stage in vitro and whose kinetics of development is compared to that of embyros obtained in vivo.

This evaluation and/or the determination of asynchrony of development T is carried out preferably by cell counting or determination of the portion of embryo cells organized into an internal cellular mass, which cells will contribute to the formation of the fetus and of part of the placenta. Nevertheless, other technologies known to a person skilled in the art can be used to carry out this evaluation and/or determination, such as for example the demonstration of the expression and/or of the absence of expression of cellular markers which are characteristic of a particular stage of embryonic development.

The asynchrony of development T is preferably greater than or equal to 15 hours, preferably greater than or equal to 16 hours, greater than or equal to 17 hours, greater than or equal to 18 hours, greater than or equal to 19 h 00 min, greater than or equal to 20 h 00 min, greater than or equal to 21 h 00 min, greater than or equal to 22 h 00 min, greater than or equal to 23 h 00 min, greater than or equal to 24 h 00 min, greater than or equal to 25 h 00 min, greater than or equal to 26 h 00 min, greater than or equal to 27 h 00 min, greater than or equal to 28 h 00 min, greater than or equal to 29 h 00 min or greater than or equal to 30 h 00 min. More preferably, said asynchrony of development T is about 24 hours.

In the method according to the present invention, said embryo transferred in step b) is cultured under the same conditions as said first embryo. According to a first preferred embodiment, said embryo transferred in step b) is at the 1 cell stage. According to a second embodiment, said embryo transferred in step b) is at the 2 cell stage. According to a third embodiment, said embryo transferred in step b) is at the 4 cell stage. Alternatively, said embryo transferred in step b) is at the 8 cell stage, at the 16 cell stage, at the 32 cell stage, at the 64 cell stage or at a more advanced stage of development.

The methods for producing vasectomized male animals are well known to a person skilled in the art, as well as the hormone treatments intended to increase ovulation (see for example: Kennely and Foote, 1965).

Said embryo transferred in step b) of the method according to the invention develops into a fetus, preferably, said fetus develops into a newborn, and said newborn develops into an adult. It is therefore also one of the objects of the present invention to provide an embryo, a fetus, a newborn, an adult animal, except humans, or cells derived therefrom, produced by a method comprising or including the method according to the invention. The invention also relates to the progeny of said adult mammal according to the invention. For ethical reasons, it is obvious that the method according to the invention must not be carried out for the purpose of reproductive cloning of human beings.

Depending on the needs, it may be advantageous to stop the development or the gestation of the embryo at the embryonic or fetal stage in order to derive cells, in particular cells of the internal cellular mass, such as stem cells, from said embryo. The expression stem cells of the embryo is understood to mean the pluripotent undifferentiated cells, which can be cultured in vitro in a prolonged manner without losing their characteristics, and which are capable of differentiating into one or more cell types when they are placed under defined culture conditions. Thus, when the stem cells according to the invention are ES cells, it is possible to envisage inducing the differentiation thereof into different types of cell such as for example muscle, cardiac, glial, nerve, epithelial, hepatic, pulmonary or pancreatic cells. Thus, in the context of a so-called “therapeutic” cloning, the embryo may be a human embryo obtained by an intra- or interspecies nuclear cloning method so as to obtain stem cells, differentiated or not, which are useful for the preventive or curative treatment of patients requiring such a treatment. In this case, steps b) for transferring the embryo into the uterus and c) for implantation of the transferred embryo, of the method according to the invention, are optional. Persons skilled in the art know the techniques for in vitro culture of cells of the internal cellular mass (see for example WO 9737009) and of embryonic stem cells in particular, so that the latter preserve their totipotency or their pluripotency in culture (Evans et al., 1981; EP 380,646; WO 9730151) or so that the latter induce their differentiation into a particular cell type.

The subject of step b) of the method according to the invention is the development of a nonhuman animal from the embryonic stage to full term. This can be done directly or indirectly. In a direct development, the reimplanted embryo, which is transgenic or not, reconstituted or not, is simply left to develop in the uterus of the surrogate mother without any outside intervention up to full term. In an indirect development, the embryo can be manipulated before complete development has taken place. For example, the embryo can be divided, and the cells developed clonally, with the aim of increasing the yield of production of cloned animals. Alternatively or additionally, it is possible to increase the yield of production of viable embryos by the successive use of the nuclear transfer method according to the invention.

The embryo, fetus, newborn, adult mammal, except humans, or cells derived therefrom, obtained using the method according to the invention, may also be transgenic. Transgenesis is either carried out during in vitro culture of the embryo, or the embryo is derived from an animal which is itself transgenic. For the purposes of the present invention, the expression “transgenic” is understood to mean a cell or an animal containing at least one transgene. The expression “transgene” is understood to mean genetic material which has been or which will be artificially inserted into the genome of a cell of an animal according to the invention, particularly into a mammalian cell cultured in vitro or into a cell of a live mammal and which will be maintained therein in said cell in episomal form. The methods for generating transgenic cells according to the invention are well known to a person skilled in the art. They include, without being exhaustive, the technology of targeted inactivation of one or more genes by homologous recombination (“Knock-Out”), the technology of targeted insertion of one or more genes by homologous recombination (“Knock-In”), the technology of random integration of a transgene by microinjection into the nucleus. The transgene according to the invention, optionally contained in a vector which is linearized or not, or in the form of a vector fragment, may be introduced into the host cell by standard methods such as for example microinjection into the nucleus (U.S. Pat. No. 4,873,191), transfection by calcium phosphate precipitation, lipofection, electroporation, heat shock, transformation with cationic polymers (PEG, polybrene, DEAE-dextran and the like), viral infection and sperm.

The reimplantation of the embryo into the uterus of a surrogate female uses techniques known to a person skilled in the art. Normally, the surrogate mother is anesthetized and the embryos are inserted into the oviduct. The number of embryos implanted into a particular host varies according to the species but is normally compatible with the number of newborns normally produced by said species. The transgenic or nontransgenic progeny of the surrogate mother are screened for the presence and/or expression of the transgene or of a marker characteristic of said progeny using suitable methods. The screening is often carried out by Southern blotting or by Northern-blot analysis using a probe which is complementary to at least a portion of the transgene or of the marker. Western-blot analysis using antibodies to the protein encoded by the transgene or said marker may be used as an alternative or as an additional method for screening for the presence of the protein product encoded by the transgene or said marker. Typically, the DNA is prepared from cells of the animal, and in particular the lymphocytes in rabbits, and then analyzed by Southern blotting or by PCR for the presence of the transgene. Alternatively, the tissue of the cells capable of expressing the transgene or the marker at the highest level are tested for the presence and/or expression of the transgene or marker using Southern-blot analysis or by PCR. Alternatively, additional methods for evaluating the presence of the transgene or the marker are biochemical methods, such as enzymatic and/or immunological tests, histological methods to make it possible to detect the presence of a particular marker or of certain enzymatic activities, and flow cytometry analyses.

It is also one of the objects of the present invention to provide an in vitro method of cloning the mammal by nuclear transfer comprising or including a method according to the invention for producing nonhuman mammalian embryos which comprises at least the step of evaluating and/or determining the asynchrony of development T between two embryos of the same species and of the same age. The expression nuclear transfer or nucleus transfer is understood to mean the transfer of a nucleus from a donor cell obtained from an animal according to the invention, preferably from a mammal, at a stage of development between the embryonic stage and the adult stage, into the cytoplasm of an enucleated recipient cell of the same species or of a different species. In general, the recipient cell is an oocyte. The transferred nucleus is programmed to direct the development of the cloned embryos which can then be transferred into the uterus of surrogate females in order to produce fetuses and newborns, or used to produce cells of the internal cell mass in culture.

The donor genetic material is introduced by various means into the enucleated recipient cell so as to form the reconstituted embryo. Generally, the donor genetic material is introduced by fusion using methods such as (i) exposure of the cells to chemical agents promoting fusion such as ethanol, polyethylene glycol (PEG) or other glycols; (ii) use of biochemical agents, such as phytohemagglutinin (PHA); (iii) use of inactivated viruses, such as Sendai virus; (iv) use of liposomes; (v) use of electrofusion. The present invention is not limited to the use of these fusion techniques and although cell-cell fusion is the preferred method for carrying out nuclear transfer (McGrath and Solter, 1984; WO 9937143), other methods which are also preferred can be used, such as microinjection, preferably microinjection of the donor nucleus (Wakayama et al., 1998).

According to the present invention, the donor cell and the recipient cell, preferably an oocyte, are obtained from the same animal, or from two animals of the same species. According to another embodiment, the donor cell and the recipient cell are obtained from two animals of different species.

The combination of the genome of the activated donor cell and of the activated oocyte produces the embryo obtained by nuclear transfer, or NT (for “nuclear transfer”) embryo, also called reconstituted embryo, which terms will be used interchangeably in the present patent.

The donor cell according to the invention may be any cell type which contains a genome or genetic material, such as somatic cells, germ cells, embryonic cells such as pluripotent stem cells, totipotent stem cells, such as embryonic stem cells for example (ES cells). The term “somatic cell” refers to differentiated diploid cells. The somatic cell may be obtained either from an animal, or from a cell or tissue culture which has undergone at least one passage in culture and which has been frozen or not. When the somatic cell is derived from an animal, the animal may be at any stage of development, for example an embryo, a fetus or an adult. The somatic cells preferably comprise, and without limitation, fibroblasts (for example primary fibroblasts), epithelial cells, muscle cells, cumulus cells, neural cells, mammary cells, hepatocytes, Langerhans' cells. Preferably, the donor somatic cells are cumulus cells. The somatic cells may be obtained for example by dissociating tissues by mechanical or enzymatic means (in general by the use of trypsin or of proteases) so as to obtain a cellular suspension which is generally cultured until a confluent cell monolayer is obtained. The somatic cells may be harvested or prepared for cyropreservation and maintained frozen until subsequent use. The nucleus donor cells are either in the proliferative state or in the quiescent state. The quiescent state which corresponds to the G0/G1 stage of the cell cycle is obtained in cells in culture by contact inhibition or by serum deprivation (Whitfield et al., 1985). The proliferative state may be considered as corresponding to all the other stages of the cell cycle.

The recipient cells according to the present invention are preferably oocyte, more preferably activated oocytes. The activated oocytes are those which are at a stage of meiotic cell division which comprises the prophase, the anaphase, the metaphase, the telophase I and II, preferably the metaphase I, the anaphase I, the anaphase II, and preferably the telophase II. The invention also relates to the oocytes in metaphase II which are considered as being in a resting state, but which may be activated by techniques known to a person skilled in the art (WO 0025578). The state of development of the oocyte is defined by visual inspection of the oocyte at a sufficient magnification. The oocytes which are in telophase II are for example identified by the presence of a protrusion of the plasma membrane corresponding to the second polar globule. The methods for identifying the various stages of meiotic cell division are known to a person skilled in the art.

Various techniques have been described for activating the oocytes, such as the use of calcium ionophores (for example ionomycin) (see U.S. Pat. No. 5,496,720) which are agents which increase permeability of the membrane of the oocytes and allow calcium to enter into the oocytes. Also, ethanol, which has the same effects, can be used. Also, the activation of the oocytes may be carried out using sets of electrical stimulations which may be used in rabbits to control the level of calcium in the oocytes (Ozil and Huneau, 2001). Preferably, the activation of the ooctyes is obtained by a set of electrical pulses, and then chemically extended by culturing the oocytes in the presence of a protein kinase inhibitor such as 6-dimethylaminopurine (6-DMAP) and/or in the presence of an inhibitor of peptide synthesis such as cycloheximide (CHX). The step of activating the oocyte may be carried out before, during and/or after the step of fusion of the nucleus or of the donor cell with the recipient oocyte.

The oocytes may be obtained by maturation in vitro of material obtained by follicular puncture of ovaries collected in slaughter houses or by aspiration of the oocytes from the follicles of ovaries at defined moments of the reproductive cycle of a female who has or has not been hormonally stimulated exogenously (superovulated female). The oocytes are matured in vivo or in vitro up to the metaphase II or telophase stage. All the oocytes matured in vivo must be harvested by washing the oviduct in a PBS (phosphate buffered saline) buffer. The in vitro matured oocytes are collected and transferred into a culture medium containing 10% serum, such as fetal calf serum (FCS). The oocytes are freed of cumulus cells and then enucleated as previously described by Adenot et al. (1997).

The present invention relates more particularly to a method for producing rabbit embryos comprising the following steps:
(a) evaluate and/or determine the asynchrony of development (T) between two rabbit embryos of the same age:
   the first embryo being produced by crossing at the time t₀ a male, preferably vasectomized, with a female who has preferably received hormone treatment to increase ovulation, said first embryo being at least cultured and/or manipulated in vitro;
   the second embryo being produced by crossing at the time t₀ a fertile male with a female who has preferably received hormone treatment in order to increase ovulation, said second embryo being normally fertilized and obtained by parthenogenetic activation,
the evaluation and/or determination taking place at the latest on the day of uterine implantation of said second embryo normally fertilized or obtained by parthenogenetic activation; and
(b) transfer a rabbit embryo which is cultured and/or manipulated in vitro, no older than the blastocyst stage into the uterus of a recipient female who was crossed with a vasectomized male at the time t=t₀+T (±25% T);
(c) optionally, allow said embryo transferred in step b) to become implanted and to develop in the uterus of said recipient female.

The evaluation and/or determination is carried out at a stage of development between days D1 and D10 post coitum, preferably between days D1 and D8 post coitum. More preferably, the evaluation and/or determination is carried out in vitro on days D3 and D4 post coitumn.

In the method for producing rabbit embryos according to the invention, said asynchrony of development T is 23 hours ±25%. Preferably, said asynchrony is about 20 hours, about 21 hours, about 22 hours, about 23 hours or about 24 hours.

According to a preferred embodiment of the invention, said rabbit embryo cultured and/or manipulated in vitro is a transgenic embryo. According to another preferred embodiment, said embryo cultured and/or manipulated in vitro is a reconstituted embryo obtained by nuclear transfer. More preferably, said embryo cultured and/or manipulated in vitro is a reconstituted transgenic embryo obtained by nuclear transfer.

In the method for producing rabbit embryos according to the present invention, said embryo transferred in step b) is preferably at the 1, 2 or 4 cell stage, although subsequent stages of development may be envisaged.

The rabbit embryos and/or fetuses, newborns, adult rabbits, progeny of adult rabbits, or cells derived therefrom, produced by a method comprising or including the method for producing rabbit embryos according to the invention are also the subject of the present invention.

The invention also relates to an in vitro method for cloning rabbits by nuclear transfer, comprising or including a method for producing rabbit embryos, which are preferably transgenic, according to the invention. More particularly, this in vitro method for cloning rabbits by nuclear transfer corresponds to the steps of:
a) inserting a rabbit donor cell or a rabbit donor cell nucleus into a rabbit enucleated oocyte under conditions which make it possible to obtain a reconstituted embryo;
b) activating the reconstituted embryo obtained in step a);
c) transferring said reconstituted embryo into a surrogate rabbit, such that the reconstituted embryo develops into a fetus, and possibly into a newborn;
and is characterized in that the method comprises or includes a method for producing rabbit embryos according to the invention. Preferably, the transfer of nucleus into the recipient cytoplasm is carried out by fusion of the donor cell and the recipient cytoplasm; alternatively, the transfer of nucleus into the recipient cytoplasm is carried out by microinjection of the donor nucleus into the recipient cytoplasm.

To successfully carry out nuclear cloning in animal species which have up until now been excluded from such technology, such as for example rabbits and rats, or in animal species which are difficult to clone, it is also important that the activation phase in vitro of the reconstituted embryo is as brief as possible. Indeed, in species such as rabbits, this S phase occurs very rapidly compared with other species (Szöllösi, 1966). The shortening of the activation procedure allows the DNA replication phase (S phase) of the first cell cycle to occur, in relation to ovulation, at a moment which is identical to that for normal development. The present invention therefore provides means for reducing the phase for in vitro activation of the reconstituted embryo, so as to ensure a sufficient kinetics of development so that the embryo does not find itself outside the implantation window even after desynchronization. The inventors have thus demonstrated, quite surprisingly, that the mixture of two drugs, one being a protein kinase inhibitor, such as 6-dimethylaminopurine (6-DMAP), and of at least one inhibitor of protein synthesis, such as cycloheximide (CHX), which have been used separately up until now to carry out the activation of reconstituted embryos, makes it possible to obtain a more efficient activation over shorter periods while limiting the known side effects of these drugs, in particular on the initiation of the S phase and the replication of DNA. Accordingly, in the method according to the invention, the activation phase during the culture in vitro is carried out by adding, preferably simultaneously, or successively or spaced out over time, to the culture medium for said reconstituted medium, at least one protein kinase inhibitor and at least one inhibitor of protein synthesis. Preferably, said activation is carried out by simultaneous addition of 6-DMAP and of cycloheximide (CHX). The 6-DMAP and CHX concentrations for carrying out such an activation in rabbits are respectively between 1 and 5 millimolar (mM), preferably 2 mM and 1 to 10 micrograms (μg), preferably 5 μg per ml. The duration of activation preferably extends from 30 minutes to 2 hours, preferably one hour. Persons skilled in the art will adjust these parameters without difficulty according to other mammals than rabbits.

The present invention therefore also relates to a method for activating an embryo which is reconstituted or not, transgenic or not, characterized in that it comprises the step of adding to the culture medium for said embryo, successively, simultaneously or spaced out over time, at least one inhibitor of protein kinase, which is more particularly involved in the resumption of meiosis, and preferably 6-DMAP, and at least one inhibitor of protein synthesis, preferably cycloheximide (CHX).

The present invention also relates to an in vitro method for cloning a mammal as described above, comprising the steps: (i) of inserting a donor cell or a donor cell nucleus into an enucleated oocyte of a mammal of the same species or of a different species from that for the donor cell, under conditions which make it possible to obtain a reconstituted embryo; (ii) of activating the reconstituted embryo obtained in step (i); and of (iii) transferring said reconstituted embryo into a mammalian surrogate female, so that the reconstituted embryo develops into a fetus, characterized in that said activation is carried out by adding successively, simultaneously or spaced out over time, to the culture medium for said reconstituted embryo, at least one protein kinase inhibitor, preferably 6-DMAP, and at least one inhibitor of protein synthesis, preferably cycloheximide (CHX). Preferably, said activation is carried out by simultaneous addition of 6-DMAP and CHX for a period of activation which preferably extends from 30 minutes to 2 hours, preferably one hour.

It is also one of the objects of the present invention to provide a method for producing a recombinant protein by a transgenic animal, in particular rabbits, obtained by a method according to the invention. The transgene which encodes said recombinant protein is not limited to a particular DNA sequence. The DNA sequence of the transgene may be of purely synthetic origin (for example routinely produced from a DNA synthesizer), or may be derived from mRNA sequences by reverse transcription, or may be derived directly from genomic DNA sequences. When the DNA sequence is derived from RNA sequences by reverse transcription, it may or may not contain all or some of the noncoding sequences such as introns, depending on whether the corresponding RNA molecule has or has not undergone, partially or completely, splicing. The transgene may be as small as a few hundredths of cDNA base pairs or as large as some hundred thousands of base pairs of a gene locus comprising the exon or intron coding sequence and the regulatory sequences necessary for obtaining controlled expression in a spatio-temporal manner. Preferably, the recombinant DNA segment has a size of between 2.5 kb and 1000 kb. Whatever the case, the recombinant DNA segments may be less than 2.5 kb and greater than 1000 kb. The transgene or the DNA sequence of the present invention is preferably in native form, that is to say derived directly from an exogenous DNA sequence which is naturally present in an animal cell. This DNA sequence in native form may be modified, for example, by inserting restriction sites necessary for the cloning and/or by inserting site-specific recombination sites (lox or flp sequences). Alternatively, the DNA sequence of the present invention may have been artificially created in vitro by recombinant DNA techniques, by combining for example portions of genomic DNA and of cDNA.

The transgene which encodes said recombinant protein preferably contains appropriate regulatory sequences for directing and controlling the expression of genes encoding said polypeptides in the appropriate cell type(s). The expression elements controlling gene expression is understood to mean all the DNA sequences involved in the regulation of gene expression, that is to say mainly the sequences for regulating transcripion, splicing and translation. Among the DNA sequences for regulating transcription, there may be mentioned the minimal promoter sequence, the upstream sequences (for example the SP1 box, the IRE for “interferon responsive element”, and the like), activating sequences (“enhancers”) optionally inhibitory sequences (“silencers”), insulating sequences (“insulators”), splicing sequences. The elements controlling gene expression allow either constitutive, ubiquitous or inducible expression which is specific for a cell type (“tissue-specific”) or specific for a developmental stage. These elements may or may not be heterologous to the organism, or may or may not be naturally present in the genome of the organism. It is obvious that depending on the desired result, persons skilled in the art will choose and will adapt the elements for regulation of the expression of genes. To direct the expression of the transgene in a biological fluid of the animal, such as milk, the sequence for regulating transcription which is used is selected from the promoter sequences of the genes which are specifically active in cells secreting these biological fluids, such as the cells of the mammary glands, for example in order to direct the expression in milk. Among the preferred biological fluids, there may be mentioned milk, blood, sperm, urine. Preferably, the recombinant protein according to the invention is secreted by the cells of the mammary glands in milk. Thus, the preferred promoter sequences or promoters are those which are both effective and specific in the mammary tissue. The expression effective is understood to mean that the promoters are strong in the mammary tissues and can support the synthesis of a large quantity of protein secreted in milk. Among these promoters, there may be mentioned the caseine, lactoglobulin and lactalbumin promoters, which include, without limitation, the α-, β- and γ-caseine promoters, the α-lactalbumin promoter and the β-lactoglobulin promoters. The preferred promoters are obtained from rodents, mice or rats, rabbits, pigs, goats, sheep. More preferably, the promoter is that of a gene for whey acidic protein (WAP) and the WAP promoter most preferred is a rabbit WAP promoter described in U.S. Pat. No. 5,965,788, the pig WAP promoter and the mouse WAP promoter.

The use of a transgenic animal, in particular a transgenic rabbit, obtained by a method according to the invention for the production of recombinant proteins of interest, preferably in the milk of the animal, is a subject of the present invention. The recombinant protein of interest may be any protein, for example a therapeutic protein such as α-, β-, δ-globin, blood coagulation factors (factors VIII and IX), cell surface receptors, antibodies, enzymes, and the like, and other proteins necessary, for example, for correcting inherited or acquired defects in a patient.

The invention also relates to the use of a transgenic animal, preferably a transgenic rabbit, which can be obtained by the method according to the invention, as a model for studying human pathologies. By way of example of human pathologies, there may be mentioned cystic fibrosis, atherosclerosis, cancers, metabolic diseases, ocular pathologies. Taking into account the genetic polymorphisms present in the population, it may be advantageous, in order to analyze or obtain a characteristic physiological, physiopathological or behavioral response, that the transgenic animals according to the invention, and in particular the transgenic rabbits according to the invention, have different genetic backgrounds. Thus, the rabbits according to the invention may be selected from the New Zealand, Fauve-de-Bourgogne, Argenté-de-Champagne, Californian and Géant-de-Bouscat breeds, any breeds whose zoological specificities are defined in an official standard (Le lapin de race, Ed. 2000 Fédération Francaise de Cuniculture (Ed.) and their crosses, in particular those which give rise to commercial strains such as the strain GD22/1077.

Other characteristics and advantages of the present invention will be demonstrated more clearly on reading the following examples.

### BRIEF DESCRIPTION OF THE DRAWINGS

FIG. 1: Confocal images of a reconstituted embryo (nt) at the 1 cell stage immunolabeled using an anti-alpha-tubulin antibody (green) and dna with propidium iodide (red).

(A). Before the second round of electrostimulation, chromatin appears condensed in the chromosomes and attached to the spindle; the arrows in the inset (view magnified 3 times of the region of the spindle) indicates individual chromosomes close to the poles of the spindle. (B). Following removal of CHX and of 6-DMAP, 72% of the reconstituted embryos (NT) (n=25) already exhibit a small nucleus and a formed interface microtubular network (arrow). (C). 1 hour after removal of the drugs, all the reconstituted embryos (NT) are in the interphase and 71% (n=17) of them have a single and large nucleus resembling the pronucleus as observed in the rabbit zygotes. Bar=50 μm.

FIG. 2: Development of rabbit reconstructed blastocysts with cumulus cells or derived from eggs activated in vitro or fertilized in vivo.

(A), Increase in vitro of the number of cells between days D3 and D4 of embryos recovered either directly from donors (in vivo, n=27) or cultured from the one-cell stage (about 20 hours post HCG), or after natural mating (in vitro, n=44) or by nuclear transfer (n=31, NT); (B), mean diameter and mean length of the embryonic disks on day D8; (C), example of reconstructed blastocyst (obtained by nuclear transfer) delayed and recovered on day D8 after a transfer at the 4-cell stage into an asynchronous recipient female (−16 hours); the embryonic disk (large arrow) is visible but the blastocyst is still surrounded by a fine layer for protecting the embryo (small arrow) which should normally have disappeared on day D7 (Denker, 1981). Embryos fertilized in vivo are harvested either directly from donors (in vivo) or following transfer into recipient females at the 1 cell stage (control). The embryos fertilized in vitro are collected at the 1 cell stage (in vitro).

FIG. 3: Schematic representation of the protocol used for the in vivo development of the reconstituted embryos from cumulus donor cells. Only the embryos transferred at the 4 cell stage into asynchronous females (at 22 hours) develop to full term.

FIG. 4: Rabbits born through nuclear transfer. (A), cloned rabbit No. 0107 with the corresponding controls: A1, expression of the autofluorescent protein eGFP (arrowhead) detected by confocal microscopy from hair follicles obtained by an ear biopsy at 1 month; A2, likewise, but the detection is carried out by light transmission microscopy; A3, amplification of the eGFP transgene (PCR 2) and of exon 10 of the CFTR gene used as a control for the quality of the DNA (PCR 1); this confirms that the rabbit No. 0107 (lines 8 and 9) and its progeny No. 107B (which died 1 day after birth; lines 10 and 11) are derived from donor cumulus cells (lines 12 to 13); B1-B2, 3 other rabbits from 2 other different litters; the rabbits in B1 have now proved that they are fertile.

### EXAMPLES

### Example 11

### Materials and Methods

1.1) Source of the Oocytes and of the Cumulus Cells

Metaphase II (MII) oocytes are collected from superovulated “New Zealand” rabbits by injections of FSH hormones followed by an injection of HCG hormone, mated with a vasectomized male 16 hours after injection of human choriogonadotrophin (hCG). The oocytes are then incubated in 0.5% hyaluronidase for 15 min (Sigma) in order to remove the cumulus cells by gentle pipeting. For the nuclear transfer, the oocytes are enucleated as previously described (Adenot et al., 1997). Simultaneously, cumulus cells are collected either from rabbits of “New Zealand” breed or from F1 rabbits obtained by a cross between rabbits of “New Zealand” breed crossed with rabbits of “Fauves de Bourgogne” breed or “New Zealand” transgenic F1 female rabbits containing a DNA construct with a sequence encoding the enhanced green fluorescence protein (eGFP) placed under the control of an EF1 (“elongation factor 1”) promoter or of an HMG promoter. The eGFP fluorescence and the PCR amplification reactions are used as markers for the cumulus donor cells. The cumulus cells are then stored at 38° C. in PBS without calcium or magnesium supplemented with 1% PVP 40 000 (polyvinylpyrrolidone (PVP)) which are then used as nucleus donor cells.

1.2) Activation of the Oocytes and Nuclear Transfer

To reconstruct the embryos by nuclear transfer (NT), individual cumulus cells are inserted by micromanipulation into the zona pellucida of the enucleated oocytes. The embryos obtained by nuclear transfer (NT embryos) and the MII oocytes are activated in the following manner: 2 phases of electrical stimulations are applied 1 hour apart with a Grass stimulator (3 pulses of continuous current of 3.2 kV×cm⁻¹ for 20 μs each in 0.3 M mannitol containing 0.1 mM of Ca²⁺ and of Mg²⁺), the first phase inducing the fusion of the oocyte and of the cumulus cell. The reconstituted embryos are maintained for one hour in a culture medium at 38° C. After the second phase, the NT embryos are incubated in the presence of cycloheximide (5 μg/ml) and of 6-DMAP (2 mM) in M199 medium for 1 hour; the oocytes are incubated with one of these drugs or both simultaneously for 1 hour. After extensive washing to remove the drugs, the cells are again placed in culture in a microdrop of 50 μl of B2 medium supplemented with 2.5% fetal calf serum (FCS) in mineral oil (Sigma M8410) at 38° C. under an atmosphere saturated with 5% CO₂. This activation protocol is applied to the NT embryos about 18 to 20 hours after mating of the donors.

1.3) Analysis of the Preimplantatory Stages

The microtubular organization and the chromatin in the NT embryos at the 1 cell stage are observed as previously described (Adenot et al., 1997), except that the fixing state lasts for 20 min at 37° C. and that the mounting medium is Vectashield (Vector Laboratories). The rates of cleavage of the NT embryos and of the parthenotes are evaluated from 21 hours to 23 hours after electrostimulation. The rates of development up to the blastocyst stage are estimated after an in vitro culture of 3 to 4 days. For the evaluation of the number of cells, the embryos are fixed as previously described, and then stained with Hoechst 33442 at 1 μg/ml and then mounted on slides with wells in Vectashield and analyzed by epifluorescence. The controls are blastocysts which either developed in vitro from a 1 cell embryo collected from a superovulated rabbit or which developed in vivo from nonsuperovulated females crossed with a male and then sacrificed 3 or 4 days later.

1.4) Development in vivo:

The recipient females are crossed with a vasectomized male either at the same time or 16 hours or 22 hours after crossing the oocyte donor females with a vasectomized male. In the case of synchronous females (that is to say crossed at the same time as oocyte donor females), the reconstituted NT embryos are transplanted at the 1 cell stage 1 to 3 hours after activation or are transplanted at the 4 cell stage after overnight culture. In the case of asynchronous recipient females (that is to say crossed 16 hours or 22 hours after the oocyte donor females), the reconstituted NT embryos are transplanted either at the 1 cell stage or at the 4 cell stage after overnight culture. The embryos are surgically transplanted through the infundibulum into each of the oviducts of the recipient females. The rate of implantation of parthenotes and of NT embryos is evaluated after sacrificing the recipient females on day D8. Pregnancy is determined by palpation 13 or 14 days after transplantation of the embryos and the pregnant recipient females are delivered by Caesarean 31 days after mating.

1.5) PCR Analysis

The presence of transgenic markers GFP is detected by PCR using a sense primer (SEQ ID NO:1) and an antisense primer (SEQ ID NO:2) (GENSET, France). To check the quality of the DNA, PCR is carried out on 300 to 400 ng of DNA prepared with the tissue extraction kit (QIAGEN, USA) with the sense primer (SEQ ID NO:3) and the antisense primer (SEQ ID NO:4) which covers exon 10 of the rabbit CFTR gene (GENSET, France). The amplifications are carried out with Taq polymerase (Q.BIOGEN, France) through 35 amplification cycles, in the following manner: 94° C. for 20 s, 57° C. for 30 s and 72° C. for 1 min. The size of the amplified fragments is 240 base pairs for the CFTR gene and 350 base pairs for the eGFP transgene. The PCR fragments are separated on a 1.5% agarose gel in-TAE (Tris acetate EDTA) and then stained with ethidium bromide and are compared to a 100 base pair ladder used as size marker (BIOLABS, England). The negative controls consist of double distilled water and DNA from the recipient female, while the positive control corresponds to DNA from cultured transgenic fibroblasts.

### Example 2

2) Activation, Nuclear Appearance and Development in vitro of the Reconstituted Rabbit Embryos Obtained by Nuclear Transfer

The inventors have previously described (Adenot et al., 1997) that rabbit ovulated oocytes aged in the oviducts before their collection (aging in vivo) form pronuclei for a period of one hour after activation by a stimulus: this corresponds to a time 3 times faster than when the freshly ovulated oocytes are cultured up to the same age (aging in vitro).

When the oocytes are activated in the presence of the inhibitor of protein phosphorylation (6-DMAP), these oocytes form pronuclei more rapidly than would oocytes aged in vivo (unpublished data from the inventors). Thus, the activation conditions can substantially alter the timing of the nuclear formation of the rabbit zygotes. By contrast to other mammalian species, rabbit zygote has the characteristic feature of entering into the S phase very early after activation (Szöllösi, 1966). This indicates that the duration of metaphase II (MII) up to the interphace transition should be carefully examined when an activation protocol for nuclear transfer in this species is established. In the somatic cloning of a mammal, 6-DMAP or the inhibitor of protein synthesis cycloheximide (CHX) are often used following activation of embryos obtained by nuclear transfer (NT) with agents increasing the intracellular calcium concentration. These drugs promote the inactivation of cdc2/cyclin-B and of the ERK/MAP kinases involved in arresting the oocytes at the MII stage. Cycloheximide nevertheless also inhibits the replication of DNA in the activated oocytes (Moos et al., 1996; Soloy et al., 1997) and 6-DMAP can also affect the kinase activity known to be involved in the regulation of the cell cycle (Meyer et al., 1997). The inventors have therefore considered that a 1 hour incubation with CHX and/or 6-DMAP after activation of the oocytes could be sufficient for the rabbit species. Previous, unsuccessful experiments of somatic cloning of rabbits used an incubation in 6-DMAP of 2 (Yin et al., 2000; Dinnyés et al., 2001) or 4 (Mitalipov et al., 1999) hours. In the present invention, the inventors found that the electrically activated MII oocytes, exposed for 1 hour to CHX (n=48), 6-DMAP (n=48) or to a mixture of the 2 drugs (n=130) cleave efficiently at the 2 cell stage (94%, 96% and 100% respectively). Nevertheless, they develop at the blastocyst stage significantly better when they are exposed simultaneously to CHX and to 6-DMAP (89%) or to 6-DMAP alone (79%) than to CHX alone (50%) (Table 1). These rates are higher (Dinnyés et al., 2001; Mitalipov et al., 1999) or similar (Yin et al., 2000) to those reported by other researchers who use, to treat the oocytes, 6-DMAP for 2 hours after electrostimulation (Yin et al., 2000; Dinnyés et al., 2001) or for 4 hours after electrostimulation (Mitalipov et al., 1999; Dinnyés et al., 2001) or who use ionomycin (Mitalipov et al., 1999).

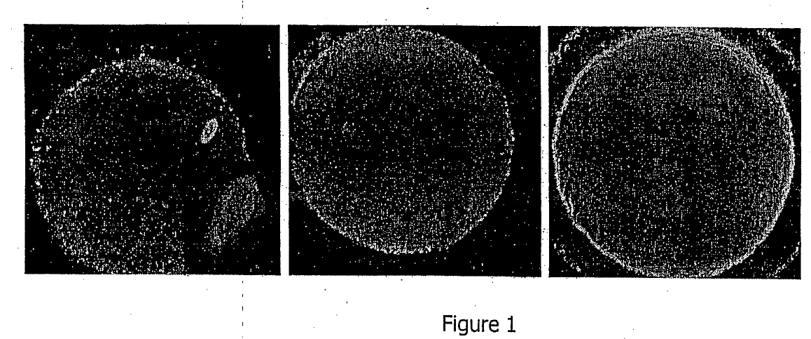

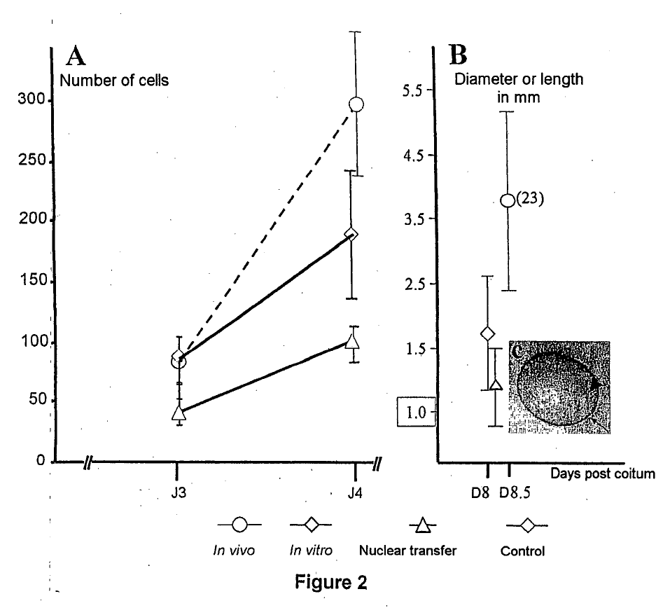

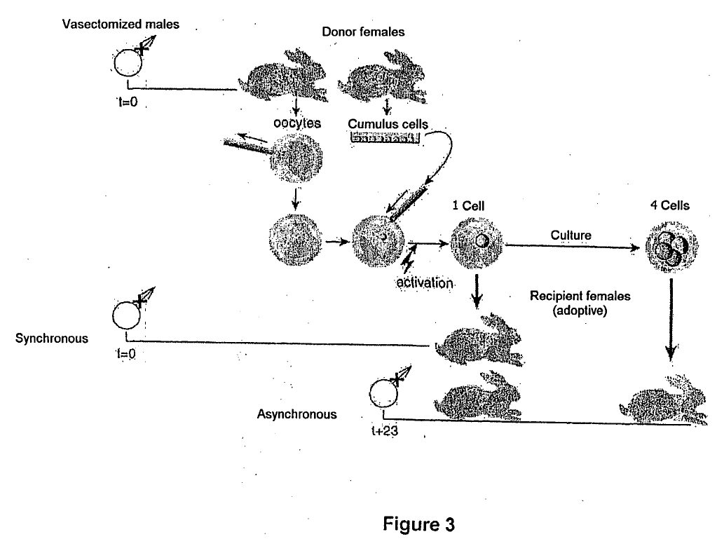

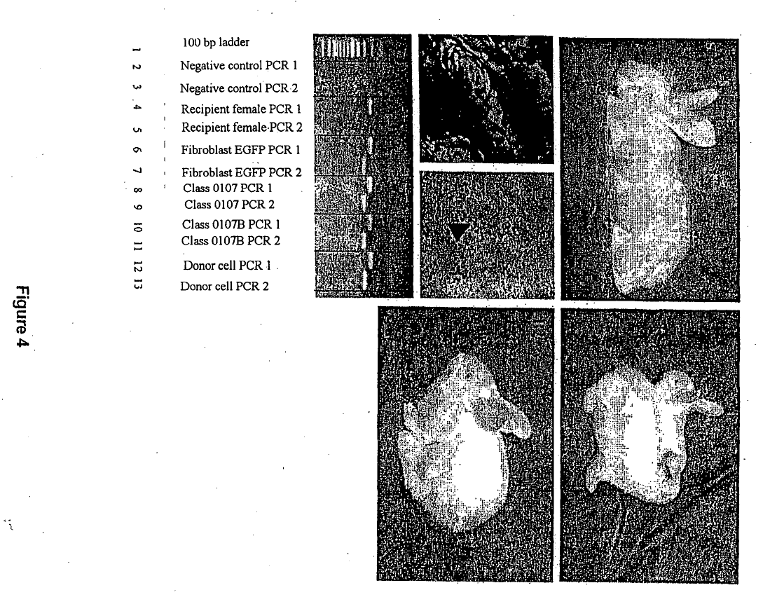

The inventors have used an activation protocol using a CHX/6-DMAP mixture to reconstruct NT embryos with a freshly collected nucleus from cumulus cells. The inventors chose this type of cells, considered as arrested at the G1/G0 stage of their cell cycle, because these cells were initially used as a model to demonstrate the feasibility of somatic cloning (Wakayama et al., 1998; Wells et al., 1999). Observations under a confocal microscope of NT embryos fixed just before the second electrostimulation phase show that the chromatin is condensed into chromosomes and associated with the spindle (N=14; FIG. 1A). This condensation results from the high MPF activity in the recipient cytoplast (Campbell et al., 1996). Whatever the case, a typical arrangement of the chromosomes characteristic of the MII oocytes was not observed. Instead of that, collapsed chromosomes form a poorly aligned metaphase plateau and individual chromosomes are sometimes observed close to the poles of the spindle (FIG. 1A, insert). This unusual chromatin structure, probably linked to the nuclear stage of the donor nucleus (Wakayama et al., 1998) is compatible with a development up to its full term in mice (Wakayama et al., 1998; Wakayama et al., 1999). After removing the drugs, the inventors observed that 72% of the rabbit NT embryos (N=25) already had an interphase chromatin and a microtubular organization (FIG. 1B). 1 hour later, all the embryos were in the interphase and 71% of them (N=17) showed a large and unique pronuclear structure (FIG. 1C) as is observed in the rabbit zygotes (data not shown). From these observations, the inventors concluded that the metaphase II to interphase transition is rapid in the reconstituted NT embryos and lead to an apparently normal remodeling of the foreign chromatin by the cytoplasm of the oocyte.

When the NT embryos are left in culture (n=135), 93% of them cleave at the two-cell stage, and 47% of them develop into blastocysts. The preimplantatory rates of development previously obtained in somatic cloning experiments in rabbits were much lower (16 to 30%) either with cumulus cells (Yin et al., 2000) or donor fibroblasts (Dinnyés et al., 2001; Mitalipov et al., 1999).

The NT embryos reach the blastocyst stage in vitro in day D3 (D3) like the zygotes and the parthenotes do, but their growth, determined by the number of cells counted, is slower, the consequence of which is that these NT embryos have a development at day D4 of about 1 day behind (FIG. 2A).

### Example 3

3) Development in vivo of Rabbit Embryos Obtained by Nuclear Transfer

In the rabbit species, the in vivo development of the blastocysts is rapid and high. This development spreads over the neighboring walls of the uterus, such that the individual position of the blastocysts becomes rapidly recognizable as sites for implantation from the D6 stage in the uterine horns of sacrificed recipient females (Denker, 1981). The inventors found that embryos obtained by nuclear transfer could form implantation sites for a transfer at the 1 or 4 cell stage into the uterus of synchronous recipient females (that is to say crossed with a vasectomized male at the same time as the nucleus donor females). However, the number of sites for implantation of the blastocysts obtained by nuclear transfer (NT) is smaller than with those of the controls and of the parthenotes (Table 2); no embryonic structure obtained by NT can be detected following dissection of the uterine horns on day D8.

When NT embryos at the 4 cell stage are transferred into asynchronous recipient females crossed 16 hours after the donor females (FIG. 3), the rate of implantation increases (Table 2) and is not significantly different from that obtained with the controls (synchronous transfer) or with the parthenotes (synchronous or asynchronous transfer). Under these conditions, the inventors were able to collect embryos at the advanced blastocyst stage; these embryos have a flat embryonic disk (FIG. 2C, large arrow) of about 1.1 mm in length (n=8, range 0.8- 1.5 mm) and all, except one, are still clearly surrounded by a fine protective layer of extracellular material (small arrow, FIG. 2C) which was gradually deposited at the surface of the rabbit embryos throughout the transit in the female genital tract. The dissolution of these mucosubstances depends on the synergistic actions of the blastocysts and of the endometrium (Denker et al., 1975) and contributes toward generating a very narrow window for implantation in this species. When these embryos are examined carefully from the abembryonic pole, no apparent disorganization of the covering can be observed, indicating that the blastocysts obtained by nuclear transfer were at least equivalent to the normal embryos at the D7 stage (Denker, 1981). None of the recipient females transplanted, either synchronously or asynchronously (16 hours) can be diagnosed as being pregnant at mid-gestation (Table 3) even when nonmanipulated “helper” embryos from another breed of rabbit (fauves de Bourgogne; data not shown) were transferred into them or when an excess of embryos obtained by nuclear transfer (up to 39 per female, data not shown) were transferred into them. These observations suggest that only very few of the blastocysts obtained by nuclear transfer can become implanted because their development is not retarded. This was confirmed by the fact that when an examination was performed on day D8, the inventors can observe in one case a blastocyst obtained by nuclear transfer already adherent to the uterine epithelium (data not shown), this blastocyst being very similar in size to the normally implanted control (range 1.1 to 2.6 mm, FIG. 2B). Although they are smaller than normal embryos in vivo at the D8 stage (Gottschewski et al., 1973) (FIG. 2B), these controls can normally develop to full term following a transfer at the 1 or 4 cell stage into synchronous recipient females (data not shown).

The inventors thus extended the asynchrony between donor females and recipient females from 16 to 22 hours (FIG. 3). Such a marked asynchrony at early cleavage stages of development has never been previously achieved with embryos obtained by nuclear transfer and is compatible with a development of zygotes to full term. Under these conditions, 10/27 (37%) of the asynchronous recipient females (22 hours) were diagnosed pregnant after palpation on day D14. Of these females, 4 (40%) gave birth on day D31 to 6 live young rabbits (FIG. 4) weighing 30 to 90 g (average weight: 65 g). Such a variability is also observed when the young rabbits are born from a small litter (from 1 to 4 fetuses) which occasionally occurs in an animal house in particular when the rabbits are obtained from embryos microinjected with DNA solutions at the pronucleus stage. The expression of the transgenic marker eGFP from hair follicles from birth (FIG. 4) and from lymphocytes obtained at the age of 1 month approximately (data not shown) confirms that the young rabbits indeed result from nuclear transfer of cumulus cells. Young rabbits having a normal morphological appearance (respective weights: 90 and 30 g) died 1 day after birth and, for one of them, the inventors suspect a deficiency in the adoption process by the nursing mother. The other 4 young rabbits developed normally and two of them (FIG. 4 in the bottom left) reproduced and gave birth to respectively 7 and 8 healthy young rabbits following a natural cross.

The present invention makes it possible to overcome the limitations encountered during the cloning of certain mammalian species which have been considered difficult to clone until now, such as rabbits (Solter 2000). These limitations can be overcome by taking into account the differences which apparently exist between oocyte development and early embryonic development. The results presented by the inventors therefore indicate that somatic cloning can be successfully carried out in any mammalian species. Surprisingly, a reduced timing, of the conventional activation procedures, and an asynchrony of nearly 1 day during the transfer of the reconstructed embryos into delayed recipient females compensate for the delay in development which already exists at the time of the first cleavage and appear to have a crucial effect on the production of rabbit embryos obtained by nuclear transfer.

The method for producing rabbits by nuclear cloning is of great industrial interest in particular for the production of transgenic rabbits expressing a protein of interest, or for the genesis of an animal model of human pathologies.

TABLE 1
Effect of various treatments on in vitro development
after parthenogenic activation of rabbit oocytes

| Treatment | | | | |
|---|---|---|---|---|
| Electrical activation | | | Number of | Number of |
| 1 hour incubation in M | | Number of | cleaved | blastocysts |
| 199+ | | oocytes used | oocytes (%) | (%) |
| CHX | 30 min | 48 | 41 (85.4) | 19 (39.6) |
| CHX | 1 h | 48 | 45 (93.7) | 24 (50.0) |
| CHX | 4 h | 48 | 47 (97.9) | 25 (52.1) |
| 6-DMAP | 30 min | 48 | 43 (89.6) | 32 (66.7) |
| 6-DMAP | 1 h | 48 | 46 (95.8) | 38 (72.9) |
| 6-DMAP | 1 h | 48 | 47 (97.9) | 35 (72.9) |
| CHX/6DMAP | 30 min | 52 | 45 (93.7) | 37 (71.1) |
| CHX/6DMAP | 1 h | 130 | 130 (100.0) | 116 (89.6) |
| CHX/6DMAP | 1 h | 36 | 36 (100.0) | 32 (88.9) |
| (reconstituted | | | | |
| oocytes) | | | | |

(CHX: cycloheximide;
6-DMAP: 6-dimethylaminopurine)

TABLE 2
Implantation after transfer into synchronous and
asynchronous (−16 hours) recipient females

| | | | Number of | |
|---|---|---|---|---|
| | | Recipient | implantation | |
| | | females | sites/number of | Number of |
| | | pregnant on | embryos | blastocysts |
| | | day D8/total | transferred | implanted/ |
| | Type of | of recipient | into pregnant | number of |
| Type of | recipient | females | recipient | recipient |
| embryos | females | transferred | females (%) | females |
| Control | synchronous | 5/6 | 15/54 (27.8%) | 9/9 |
| Parthenotes | synchronous | 8/20 | 17/78 (21.8%) | 0/1 |
| NT | synchronous | 5/16 | 7/91 (7.7%) | 0 |
| Parthenotes | asynchronous | 5/9 | 15/44 (34.1%) | 3/3 |
| NT | asynchronous | 6/13 | 12/59 (20.3%) | 1/7 |

TABLE 3
Development in vivo of rabbit reconstituted embryos by
somatic nuclear transfer

| | | Asynchronous | Asynchronous |
|---|---|---|---|
| Type of recipient females | Synchronous | (−16 h) | (−22 h) |
| Stage of the embryos | 1-cell | 1-cell | 4-cell |
| Number of reconstituted | 554 | 523 | 775 |
| embryos [Number of | [19] | [18] | [27] |
| replicates] | | | |
| Number of fused | 427 | 346 | 612 |
| embryos [% obtained | [77.1] | [66.2] | [79.0] |
| from reconstituted | | | |
| embryos] | | | |
| Total number transferred | 367 | 346 | 371 |
| [% obtained from fused | [100.0] | [100.0] | [60.6] |
| embryos] | | | |
| Number of recipient | 19 | 18 | 27 |
| females transferred | | | |
| Number of recipient | 0 | 0 | 10 |
| females pregnant on day | | | [37.0] |
| D14 [of females | | | |
| transferred] | | | |
| Number of recipient | 0 | 0 | 4* |
| females having given | | | [14.8] |
| birth [% of females | | | |
| transferred] | | | |
| Number of young rabbits | | | 6 |
| born [% of embryos | | | [1.6] |
| transferred] | | | |
| Number of live young | | | 4 |
| rabbits at the time | | | |
| of weaning | | | |
| Average weight of the | | | 65 ± 20⁺ |
| young rabbits at | | | |
| birth (in g) | | | |

*abortions between days D15 and D29 of pregnancy (13 degenerated cotyledons and fetuses were recovered)
⁺average weight of the young rabbits at birth in the animal house of the inventors, for litters of equivalent number: 55.8 g ± 17.0

References
Adenot et al. (1997) Mol. Reprod. Dev. 46, 325-336.
Baguisi et al. (1999) Nature Biotechnol. 17, 456-461.
Bromhall et al. (1975) Nature 258, 719-722.
Campbell et al. (1996) Rev. Reprod. 1, 40-46.
Chen et al. (2001) Mol. Biol. Evol. 18, 1771-1788.
Denker et al. (1975) Cytobiol. 11, 101-109.
Denker (1981) Adv. Anat. Embryol. Cell Biol. 53, 123p.
Dinnyés et al. (2001) Biol. Reprod. 64, 257-263.
Evans et al. (1981) Nature 292, 154-156
Fan et al. (1999) Pathol. mnt. 49, 583-594.
Gottschewski et al. (1973) Schaper, Hannover.
Graur et al. (1996) Nature 379, 333-335.
Hoeg et al. (1996) Proc. Natl. Acad. Sci. USA 93, 11448-11453.
Kennely and Foote (1965) J. Reprod. Fert. 9, 177-188
McGrath and Solter (1984) Science 226, 1317-1319.
Meyer et al. (1997) Methods Enzymol. 283, 113-128.
Mitalipov et al. (1999) Biol. Reprod. 60, 821-827.
Moos et al. (1996) J. Cell Sci. 109, 739-748.
Ozil and Huneau (2001) Development, 128, 917-928.
Polejaeva et al. (2000) Nature 407, 86-90.
Soloy et al. (1997) Biol. Reprod. 57, 27-35.
Solter (2000) Nat. Rev., Genet. 1: 199-207.
Stinnackre et al. (1997) L.M. Houdebine ed., Harwood Academic Publishers, pp. 461-463
Szolldsi (1966) Anat. Rec. 154, 209-212.
Wakayama et al. (1998) Nature 394, 369-374.
Wakayama et al. (1999) Proc. Natl. Acad. Sci. USA 96, 14984-14989.
Wells et al. (1999) Biol. Reprod. 60, 996-1005.
Whitfield et al. (1985) Control of Animal Cell Proliferation 1, 331-365.
Wilmut et al. (1997) Nature 385, 810-813.
Yin et al. (2000) Theriogenology 54, 1460-1476.

1. A method for producing nonhuman mammalian embryos comprising the following steps:
(a) determining asynchrony of development (T) between two embryos of the same species and of the same age:
(i) wherein a first embryo is produced by crossing at time t₀ a vasectomized male with a female, said first embryo being cultured, manipulated in vitro, or cultured and manipulated in vitro; and
(ii) a second embryo being produced by crossing at time t₀ a fertile male with a female, said second embryo being normally fertilized and obtained by parthenogenetic activation, the determination taking place at on or before the day of uterine implantation of said second embryo;
(b) transferring an embryo which is cultured, or manipulated, or cultured and manipulated in vitro into a uterus of a recipient female who was crossed with a vasectomized male at time t=t₀+T (±25% T)+.

2. The method of claim 1, wherein said first embryo is cultured, or manipulated, or cultured and manipulated in vitro up to the day of implantation.

3. The method of claim 1, wherein said determination is carried out at a stage of development chosen from a 1 cell stage, 2 cell stage, 4 cell stage, 8 cell stage, 16 cell stage, morula stage and blastocyst stage.

4. The method of claim 3, wherein said determination is carried out at the blastocyst stage.

5. The method of claim 1, wherein said determination of the asynchrony of development T is carried out by cell counting.

6. The method of claim 1, wherein said asynchrony of development T is at least 15 hours.

7. The method of claim 1, wherein said asynchrony of development T is about 24 hours.

8. The method of claim 1, wherein said embryo transferred in step b) is cultured under the same conditions as said first embryo.

9. The method of claim 1, wherein said embryo transferred in step b) is at a 1 cell stage.

10. The method of claim 1, wherein said embryo transferred in step b) is at a 2 cell stage.

11. The method of claim 1, wherein said embryo transferred in step b) is at a 4 cell stage.

12. The method of claim 1, wherein said transferred embryo develops into a fetus.

13. The method of claim 12, wherein said fetus develops into a newborn.

14. The method of claim 1, wherein said embryo cultured, or manipulated, or cultured and manipulated in vitro is a transgenic embryo.

15. The method of claim 1, wherein said embryo cultured, or manipulated, or cultured and manipulated in vitro is a reconstituted embryo obtained by nuclear transfer.

16. The method of claim 1, wherein said embryo cultured, or manipulated, or cultured and manipulated in vitro is a reconstituted transgenic embryo obtained by nuclear transfer.

17. The method of claim 1, wherein said mammal is selected from the group consisting of rodents, lagomorphs, hoofed animals, equine animals and non-human primates.

18. The method of claim 17, wherein said mammal is a rodent selected from the group consisting of mice, rats, hamsters, and guinea pigs.

19. The method of claim 17, wherein said hoofed animal is selected from the group consisting of bovines, ovines, caprines and porcines.

20. The method of claim 17, wherein said lagomorph is rabbit.

21. A nonhuman embryo of a mammal, fetus, newborn, adult mammal, or cells derived therefrom, produced by the method of claim 1.

22. A nonhuman embryo of a transgenic mammal, fetus, newborn, adult mammal, or cells derived therefrom, produced by the method of claim 1.

23. A nonhuman embryo of a mammal reconstituted in vitro obtained by nuclear transfer, fetus, newborn, adult mammal, or cells derived therefrom, produced by the method of claim 1.

24. A progeny of said nonhuman adult mammal as claimed in claim 23.

25. An in vitro method for cloning a nonhuman mammal by nuclear transfer wherein the method comprises a step of using a nonhuman mammalian embryo according to the method of claim 1.

26. A method for producing rabbit embryos comprising the following steps:
(a) determininge asynchrony of development (T) between two same age rabbit embryos
wherein a first embryo is produced by crossing at time t₀ a vasectomized male with a female, said first embryo being cultured, or manipulated, or cultured and manipulated in vitro; and
a second embryo is produced by crossing at time t₀ a fertile male with a female, the second embryo being normally fertilized and obtained by parthenogenetic activation; said determination taking place on or before the day of uterine implantation of said second embryo normally fertilized or obtained by parthenogenetic activation;
(b) transferring a rabbit embryo which is cultured, or manipulated, or cultured and manipulated in vitro, no older than blastocyst stage into a uterus of a recipient female who was crossed with a vasectomized male at time t=t₀+T(±25% T);

27. The method of claim 26, wherein said determination is carried out at a stage of development between days D1 and D7 post coitum.

28. The method of claim 27, wherein said determination is carried out on day D5 post coitum.

29. The method of claim 26, wherein said asynchrony of development T is about 17.25 to 28.75 hours.

30. The method of claim 26, wherein said embryo cultured, or manipulated, or cultured and manipulated in vitro is a transgenic embryo.

31. The method of claim 26, wherein said embryo cultured, or manipulated, or cultured and manipulated in vitro is a reconstituted embryo obtained by nuclear transfer.

32. The method of claim 26 wherein said embryo cultured, or manipulated, or cultured and manipulated in vitro is a reconstituted transgenic embryo obtained by nuclear transfer.

33. The method of claim 26, wherein said embryo transferred in step b) is at a 1 cell stage.

34. A rabbit embryo or fetus, newborn, adult rabbit, or cells derived therefrom, produced by the method of claim 26.

35. A transgenic rabbit embryo or fetus, newborn, adult rabbit or cells derived therefrom, produced by the method of claim 26.

36. An in vitro reconstituted rabbit embryo obtained by nuclear transfer, or fetus, newborn, adult rabbit, or cells derived therefrom, produced by the method of claim 26.

37. A progeny of said adult rabbit as claimed in claim 34.

38. An in vitro method of cloning rabbits by nuclear transfer comprising the method of claim 26.

39. An in vitro method for cloning rabbits by nuclear transfer, said method comprising the steps of:
a) inserting a rabbit donor cell or a rabbit donor cell nucleus into a rabbit enucleated oocyte under conditions which make it possible to obtain a reconstituted embryo;
b) activating the reconstituted embryo obtained in step a);
c) transferring said reconstituted embryo into a surrogate rabbit, such that the reconstituted embryo develops into a fetus or newborn; whereby the method comprises or includes a method as claimed claim 26.

40. The method of claim 39, wherein the transfer of nucleus into a recipient cytoplasm is carried out by fusion of the donor cell and of the recipient cytoplasm.

41. The method of claim 39, wherein the transfer of nucleus into a recipient cytoplasm is carried out by microinjection of the donor nucleus into the recipient cytoplasm.

42. The method of claim 39, wherein said activating the reconstituted embryo is carried out by adding simultaneously, successively or spaced out over time, to culture medium for said reconstituted embryo, at least one protein kinase inhibitor and at least one inhibitor of protein synthesis.

43. An in vitro method for cloning nonhuman mammals, comprising the steps of:
a) inserting a donor cell or a donor cell nucleus into an enucleated oocyte of a mammal of a same species or of a species different from that of the donor cell under conditions which make it possible to obtain a reconstituted embryo;
b) activating the reconstituted embryo obtained in step a);
c) transferring said reconstituted embryo into a surrogate female mammal, such that the reconstituted embryo develops into a fetus, whereby said activation is carried out by adding simultaneously, successively or spaced out over time, to culture medium for said reconstituted embryo, at least one protein kinase inhibitor and at least one inhibitor of protein synthesis.

44. The method of claim 43, wherein said mammal is selected from the group consisting of rabbits, rodents, in particular rats, mice, and from bovines, ovines, caprines, porcines, equines, and nonhuman primates.

45. The method of claim 42 wherein said protein kinase inhibitor is 6-DMAP and said inhibitor of protein synthesis is cycloheximide (CHX).

46. A method for producing a recombinant protein by a transgenic animal comprising the step of producing a nonhuman mammalian embryo according to the method of claim 1.

47. A method for producing a recombinant protein by a transgenic rabbit comprising the step of producing a rabbit embryo according to the method of claim 26.

48. A method of studying human pathology wherein the method comprises a step of using a nonhuman mammalian embryo according to the method of claim 1, as a model.

49. A method of using a transgenic animal to produce recombinant proteins wherein the method comprises a step of using a nonhuman mammalian embryo according to the method of claim 1.

50. The method of use of claim 49, wherein said recombinant protein is produced in milk of the transgenic animal.

51. The method of claim 1, wherein said embryo transferred in step b) is implanted and allowed to develop in the uterus of said recipient female.

52. The method of claim 1, wherein said female has received hormone treatment to increase ovulation.

53. The method of claim 26, wherein said embryo transferred in step b) is implanted and allowed to develop in the uterus of said recipient female.

54. The method of claim 26, wherein said female has received hormone treatment in order to increase ovulation.

55. The method of claim 26, wherein said asynchrony of development T is about 23 hours.

56. The method of claim 43, wherein said protein kinase inhibitor is 6-DMAP and said inhibitor of protein synthesis is cycloheximide (CHX).
